(19) United States
(12) Patent Application Publication
(10) Pub. No.: US 2002/0172648 A1
(43) Pub. Date: Nov. 21, 2002

(54) Anti-dandruff composition and method of preventing, reducing or treating dandruff formation with same

(76) Inventors: Ursula Hehner, Brensbach (DE); Karl-Heinz Ringert, Darmstadt (DE); Hans Hoelzel, Fraenkisch-Crumbach (DE)

Correspondence Address: STRIKER, STRIKER & STENBY, 103 East Neck Road, Huntington, NY, 11743, US

(21) Appl. No.: 10/086,200

(22) Filed: Feb. 28, 2002

(30) Foreign Application Priority Data
Mar. 9, 2001 (DE) 10111288.2

(51) Int. Cl. A61K 7/06

(52) U.S. Cl. 424/70.1; 424/74

(57) ABSTRACT

The improved anti-dandruff composition contains from preferably 0.03 to 20 percent by weight of a combination of three known anti-dandruff agents, namely climbazole, zinc pyrithione and piroctone olamine. Each of these anti-dandruff agents is preferably contained in an amount of from 0.01 to 10 percent by weight. The improved anti-dandruff agent is applied to a hair-covered body surface to eliminate or alleviate existing dandruff or to prevent or reduce occurrences of dandruff.

### BACKGROUND OF THE INVENTION

The subject matter of the present invention is a composition with cleaning and care properties containing a combination of three anti-dandruff active ingredients comprising climazole, piroctone olamine and zinc pyrithione and its use for treating dandruff formation on hair-covered skin, especially on the scalp.

Many people suffer from dandruff formation. The easy appearance of dandruff on the scalp and in the hair is generally seen as an indication of poor grooming. Furthermore the itching or tingling of the skin accompanying dandruff formation is generally considered disturbing. Since the itching frequently triggers a scratching reaction, it can lead to injury of the concerned skin area. Because of that as an additional complication infections with pathogenic excitation can also occur.

The dandruff that occurs generally on the hair-covered skin, especially the scalp skin, can have different causes. Normally dandruff arises because the continuously growing uppermost scaly layer of epidermis is stripped off in the form of the smallest flakes, i.e. microflakes. These microflakes can adhere to a greater extent on the more strongly fatty scalp skin because of the sebum secreted from the sebaceous gland of the hair follicle.

In many cases however the microbial build-up or growth on the skin causes an early and increased release of the uppermost scaly layer in the form of dandruff. For example, it is known that the fungus, Pityrosporum ovale (syn. Malassezia furfur) grows to a significantly greater extent on the scalp skin of dandruff-carrying persons than on persons without dandruff (K. SCHRADER, in: Cosmetics Foundations and Formulations (Grundlagen und Rezepturen der Kosmetika), Hüthig (Editors), 2^{nd} Edition, 1989, p. 970.

Pityrosporum ovale generates free fatty acids and lipoperoxides by cleavage of the sebum of the skin and thus causes scalp itching. Because of that activity mitosis activity increases, which leads to increased corneozyte formation and thus causes an increased amount of dandruff. It is known that this process is not activated if Pityrosporum ovale is inhibited and then dandruff formation is prevented.

There are many requirements for a hair cosmetic composition, which prevents or reduces dandruff formation. On the one hand, the scalp skin and thus the hair on it should be free of dandruff. On the other hand, the composition should be compatible not only with the scalp skin, but also the hair should not be damaged when the anti-dandruff composition is applied to it. The luster and combability of the hair should be maintained with the anti-dandruff composition on it.

A series of hair cosmetic compositions are already known which help to prevent or reduce dandruff formation on the scalp skin. These cosmetic compositions are available as shampoos, lotions or hair waters or tonics.

In the case of microbial origin of the dandruff different special anti-dandruff agents are suggested for use. These anti-dandruff agents should prevent the growth of microorganisms, especially Pityrosporum ovale, on the scalp skin.

The known anti-dandruff agents include the compounds (1-(4-chlorophenoxy)-1-(1H-imidazolyl)-3,3-dimethyl-2-butanone(INCI name: climbazole, Trade name Crinipan AD), (bis(1-hydroxy-2-(1H)-pyridinethionao)zinc (INCI name: Zinc pyrithione, Trade name: zinc omadine) and 1-hydroxy-4-methyl-6-(2,4,4-trimethylpentyl)-2(1H)-pyridinone, 2-aminoethanol salt (INCI name: piroctone olamine, tradename octopirox). These compounds are known and commercially obtainable. For this purpose, for example, “International Cosmetic Ingredient Dictionary and Handbook”, Vols. 1 to 3, Seventh Edition, Washington, D.C., 1997, The Cosmetic Toiletry and Fragrance Association, can be consulted. The following compounds are also used as anti-dandruff agents: synthetic chemical compounds, such as salicylic acid, sulfur, tar preparations, undecanoic acid derivatives and hinokitol, and chemical compounds from natural sources, for example extracts of arnica, birch, burdock root, poplar, burned nettle or walnut shells.

It is disadvantageous however that a high concentration of the above-named anti-dandruff compounds must be used, in order to obtain the desired reduction or prevention of dandruff, which frequently produces undesirable effects on the skin and the hair. Furthermore it is also disadvantageous that the individual effective ingredients produce no satisfactory action. Combination of two anti-dandruff agents, for example climbazole and piroctone olamine, which are used in commercial products, do not always produce a satisfactory reduction or prevention of dandruff.

### SUMMARY OF THE INVENTION

It is an object of the present invention to provide an anti-dandruff composition that does not have the above-described disadvantages and produces both an improved reduction and alleviation of the already existing dandruff and also prevents or reduces occurrences of further dandruff formation.

According to the invention the anti-dandruff composition which attains these objectives contains the effective ingredient combination comprising 1-(1-chlorophenoxy) -1-(1H-imidazolyl)-3,3-dimethyl-2-butanone (INCI name:climbazole); bis(1-hydroxy-2-(1H)-pyridinethionato)zinc (INCI name: Zinc pyrithione) and 1-hydroxy-4-methyl-6-(2,4,4-trimethylpentyl)-2-(1H)-pyridinone, 2-aminoethanol salt) (INCI name: piroctone olamine).

Surprisingly it was established that the anti-dandruff compositions according to the invention and the further embodiments described hereinbelow better prevented or reduced occurrences of dandruff formation and lastingly treated (eliminated or alleviated) an already existing case of dandruff formation. Furthermore as an additional advantage the improved action could be obtained with reduced amounts of the individual ingredients. This synergism obtained with the combination according to the invention was not to be expected.

The compositions according to the invention can advantageously be used to prevent or reduce occurrences of dandruff formation or treat (elimination or alleviation) an already existing case of dandruff on the scalp or other hair-covered skin surface (for example, the beard region, the shoulder region, the intimate region). For this purpose they are advantageously applied in a known manner on the scalp and on the head hair and/or on appropriate body locations. Then after a comparatively short acting time they are removed again, if necessary gently, by rinsing or washing off.

The present invention also includes a method of elimination, reduction and prevention of dandruff or dandruff formation on hairy body surfaces, especially the scalp, beard region, shoulders and intimate region, which comprises using the anti-dandruff composition according to the invention.

In advantageous additional embodiments at least one additional synthetic chemical and/or natural anti-dandruff agent is added to the combination according to the invention. The additional synthetic chemical anti-dandruff agent could, for example, be salicylic acid, sulfur, a tar preparation, an undeconoic acid derivative, hinokitol, an antibiotic or an antimycotic. Th additional natural anti-dandruff agents are extracts of plant origin, for example burdock root extract, poplar extract, burned nettle extract, walnut shell extract, birch extract, willow bark extract or arnica extract.

The subject matter of the invention also includes a cosmetic method for using a combination comprising the effective ingredients, climbazole, zinc pyrithione and piroctone olamine for prevention of and reducing dandruff formation or to eliminate and alleviate already existing dandruff formed on hairy body surfaces, especially on the scalp. However this subject matter also includes a method of making or manufacture of a preparation for treatment of dandruff, which is used in a preventative treatment for preventing or reducing the occurrence of dandruff as well as for treatment of an existing case of dandruff.

With the aid of the known plate diffusion tests it was established that with Pityrosporum ovale a clearly greater preventative action was obtained with the combination of climbazole plus zinc pyrithione plus piroctone olamine than could be obtained with the individual active ingredients or was the case with a combination of just two of the effective ingredients (climbazole and piroctone olamine). In contrast a stronger preventative action against dandruff formation was obtained with the combination according to the invention than with the individual effective ingredients or with the combination of two effective ingredients. Moreover it was surprisingly found that the comparatively stronger preventative action could be obtained with effective ingredient amounts which were definitely below the amounts of effective ingredients required when either individual effective ingredients or combinations of two effective ingredients were used.

The plate diffusion test performed with the test organism Pityrosporum ovale is known in itself. The following describes an exemplary method clearly.

The test organism, Pityrosporum ovale syn. Malassezia furfur (Strain CBS 6000), was initially obtained from a lyophilized culture. In order to make an inoculate suspension lyophilized material was pre-cultured (48 hours, 42° C.) in Sabouraud nutrient media with olive oil (Fluka Art. No. 75357, Pityrosporum ovale grows only with oil additives). Subsequently two runs followed on Sabouraud 4% dextrose agar (Merck Art. Nr. 5438), on whose surface a thin olive oil film was applied with a cotton swab. Then Pityrosporum ovale material was taken by means of a cotton swab from the growth on the nutrient media, which was applied covering the concerned nutrient base plate and incubated for 48 hours at 32° C. For making the inoculate suspension the plate culture was flooded with a 5.0 ml Ringer's solution with 0.1% added Tween 80 (Merck Art. Nr. 822187), taken up with a 5 ml pipette and pipetted into a sterile test tube and homogenized well. The extinction at a photometer was adjusted to 1.0 (620 nm). The suspension obtained was used as an inoculate. 200 ml of the above-mentioned nutrient medium was inoculated with 0.5 ml of this suspension. Subsequently portions of 20 ml each were taken from this nutrient medium and each poured into petri dishes and allowed to solidify. Two opposing holes (diameter 8 mm) were punched in each plate. Then 0.1 ml of portions of shampoo compositions according to example 1 containing climbazole (2.0%), zinc pyrithione (2.0%) or piroctone olamine (2.0%) individually and/or a combination of climbazole plus octopirox (2.0% each and 0.5% each) and/or a combination of climbazole (0.5%), plus zinc pyrithione (1.0%) plus piroctone olamine (0.5%) were pipetted into each hole. The plates supplied with these test samples were closed with covers and the test samples were cultured for 48 hours at 32° C. The diameter of the inhibited area on the plate was subsequently measured.

The following table I shows the inhibition area diameter obtained according to the methods of the invention in mm (hole diameter 8 mm) according to the plate diffusion test with seven different strains of Pityrosporum ovale (Malassezia fufur) CBS 6000 and DSM 6170. The entries correspond to the diameters of the inhibited area in inches.

TABLE I
DIAMETERS (in mm) OF INHIBITED GROWTH REGIONS FOR TWO
STRAINS OF CULTURED PITYROSPORUM OVALE ACCORDING
TO THE PLATE DIFFUSION TEST

| Effective Ingredients | CBS 6000 | DSM 6170 |
|---|---|---|
| Piroctone Olamine 0.5% | 50.8 | 58.8 |
| Olimbazole 0.5% | | |
| Zinc Pyrithione 1.0% | | |
| Piroctone olamine 2.0% | 27 | 26.3 |
| Climbazole 2.0% | 33.8 | 36.3 |
| Zinc pyrithione 2.0% | 37 | 34.8 |
| Climbazole 2.0% | 33.2 | — |
| Piroctone olamine 2.0% | | |
| Climbazole 0.5% | 31.4 | — |
| Piroctone olamine 0.5% | | |

These results clearly show that the combination of the three effective ingredients, climbazole plus zinc pyrithione plus piroctone olamine is clearly significantly better than the combination of climbazole plus piroctone olamine or the individual effective ingredients.

The hair cosmetic compositions according to the invention can be provided in all common commercial administration forms. For example, they can be provided as anti-dandruff shampoos, douche baths (e.g. douche gels), care compositions (anti-dandruff care compositions), rinses (anti-dandruff rinses), hair tonics (anti-dandruff hair tonics), aqueous hair sprays, foams, gels, fixing compositions, deodorants (e.g. deodorant sticks), shaving waters, pastes or creams.

The preferred administration form is a shampoo, which is formulated with an alkyl ether sulfate surfactant or a surfactant mixture of alkyl ether sulfates. The preferred surfactants for the compositions of the invention are anionic surfactants, amphoteric surfactants, zwitterionic surfactants and/or nonionic surfactants.

The preferred preparation can be prepared and filled in all containers commonly used for comparable cosmetic products.

The combination of anti-dandruff effective ingredients according to the invention can be easily worked into every base composition for cosmetic or therapeutic products according to known methods, when the effective ingredient concentration is from 0.01 to 10.0, especially from 0.05 to 5.0, percent by weight per effective ingredient (climbazole, zinc pyrithione, piroctone olamine), in relation to the total weight of the marketed or commercially available composition. The commercially available or marketed composition according to the invention contains from 0.03 to 20.0 percent by weight, preferably from 0.1 to 10.0 percent by weight, especially preferably between 0.5 to 5.0 percent by weight, of all three effective ingredients, in relation to the total amount of the commercially available composition.

Preferred embodiments of these compositions can contain at least one additional synthetic chemical anti-dandruff ingredient and/or at last one natural anti-dandruff ingredient, for example salicylic acid, sulfur, a tar preparation, an undeconoic acid derivative, hinokitol, arnica extract, birch extract, burdock root extract, poplar extract, burned nettle extract or walnut shell extract. The amount of these active ingredients can be from 0.001 to 10.0 percent by weight, especially between 0.01 and 5.0 percent by weight, especially preferably between 0.05 to 2.0 percent by weight per effective ingredient in relation to the commercially available composition.

The compositions of the invention can contain all common known additive, auxiliary and carrier substances. For example, they can contain thickeners, such as bentonite, fatty acids, starches, polyacrylic acids and their derivatives, cellulose derivative compounds and alginate; wetting agents and emulsifiers from the classes of anionic, cationic, amphoteric, zwitterionic or nonionic surface-active substances (surfactants), for example fatty alcohol sulfates, fatty alcohol ether sulfates, alkyl sulfonates, alkyl benzene sulfonates, quaternary ammonium salts, alkyl betaines, ethoxylated alkyl phenols, fatty acid alkanol amides or ethoxylated fatty acid esters; silicone surfactants or silicone oils (for example dimethicones), alcohols, such as ethanols, propanols, isopropanols or glycerol; solvating agents, stabilizers, perfumes and/or fragrances, perfume oils, dyestuffs (for example natural dye compounds or synthetic direct-dyeing dye compounds but also coloring materials (for example fluorescein sodium salts), hair conditioning and hair care ingredients and/or care substances such as cationic polymers, lanolin derivative compounds, cholesterol, pantothenic acid and betaine, preservatives, inorganic or organic acids (e.g. phosphoric acid, acetic acid, lactic acid), bases, salts (e.g. sodium chloride), buffers (e.g. sodium citrate or sodium phosphate), consistency-imparting substances (cellulose derivative compounds) light-protective agents and/or UV-filtering, natural, partially or completely synthetic polymers (such as chitosan, FMOC-chitosan, PVP).

For example the above-described additive substances or auxiliary and carrier substances, which can be contained in the compositions according to the invention, include preservatives, such as parahydroxybenzoic acid ester (for example in an amount of from 0.05 to 2.0 percent by weight), benzoic acid and formic acid, fungicide and bacterocide ingredients (for example, 2,4,4-trichloro-2-hydroxyphenylether or methylchlorisothiazolinone), plant extracts (for example burned nettle root extract or camomile extract in an amount of from 0.1 to 2.0 percent by weight), light-protective agents (for example, p-methoxycinamic acid isoamyl ester in an amount of from 0.01 to 2.0 percent by weight), thickeners (for example, cellulose and esters of it in an amount of 0.5 to 3.0 percent by weight); hair care ingredients (for example, apple wax, Ilex resin, lanolin, beeswax, panthenol, fatty alcohols, fatty acids, triglycerides, ceramide, betaines, carnitine ester, amino acids, peptides, proteins, vitamins and mixtures thereof in an amount of from 0.1 to 2.0 percent by weight). For example, the vitamins can include vitamin C, vitamin B (Biotin) and vitamin E (tocopherols). These vitamins can be included in an amount of from 0.001 to 2.0 percent by weight. For example, the proteins included in the compositions according to the invention can include keratin in an amount of from 0.1 to 4.0 percent by weight. For example, the amino acids should be cysteine or alanine in an amount of from 0.01 to 0.5 percent by weight. The emulsifiers can especially be non-ionogenic emulsifiers, for example Plantacare or alkyl oligoglucosides of Henkel, Düsseldorf, Germany, in an amount of from 0.1 to 2.0 percent by weight. The solvating agents are for example Cremophore RH 410 or glycerol polyethylene glycoloxystearate of BASF, Ludwigshafen, Germany, in an amount of from 0.1 to 2.0 percent by weight.

Known products are used as the silicone oils, for example dimethicones (e.g. Dow Corning 200 Fluids), phenyltrimethicones (e.g. Abil AV types of Goldschmidt), cyclomethicones (e.g. Dow Corning 244 Fluid), dimethiconols (e.g. Dow Corning 1401), alkyldimethicones (e.g. Dow Corning 2502 and 2503) or aminofunctional silicones (e.g. Dow Corning 939 or 8220). The silicone oils can be used in a concentration of 0.02 to 5 percent by weight, in relation to the total amount of the marketed product.

The anionic, surfactants, cationic surfactants, amphoteric surfactants, zwitterionic surfactants or nonionic surfactants can be used individually or in combination with each other in a mixture.

The suitable anionic surfactants include alkali, alkaline earth, ammonium or alkanol amine salts of alkane sulfonates, alkyl sulfonates and alkyl ether sulfates, which can contain especially from 12 to 18 carbon atoms in their alkyl groups, especially the sodium or triethanol amine salts of lauryl or tetradecyl ether sulfates.

The suitable cationic surfactants include fatty amines, quarternary ammonium compounds, for example Genamin® CTAC or cetyltrimethyl-ammonium chloride (Hoechst, Germany), quaternary compounds of pyridines, of morpholines or of imidazolines.

The nonionic surface-active substances can include ethoxylated fatty alcohols with 12 to 18 carbon atoms and with up to 40 mol ethylene oxide per mol of fatty alcohol, such as ethoxylated lauryl, tetradecyl, cetyl, oleyl or stearyl alcohol, alone or in a mixture, as well as fatty alcohols derived from ethoxylated lanolin or ethoxylates lanolin. However fatty amine ethoxylates, fatty acid alkanol aimdes and ethoxylated sorbitan fatty acid esters are suitable as surfactants or surfactant mixtures.

For example, the following amphoteric surface-active substances are suitable as wash-active materials ingredients as additional ingreident of the hair cosmetic compositions according to the invention: N-alkylbetaines, N-alkylaminobetaines, N-alkylsulfobetaines, N-alkylaminopropionates, alkyldimethylammonium acetates and the fatty acid alkylamido betaines.

The cationic polymers are likewise known in the art and commercially obtainable. One or more cationic polymers can be included in the compositions of the invention. All cosmetically acceptable cationically charged polymers can be employed, for example Luviquat® FC 905 (copolymer of vinylimidazolium metho-chloride/vinylpyrrolidone or Polyquaternium-16) of BASF, Ludwigshafen, Germany, or Gafquat® 755 N (copolymer of vinylpyrrolidone/dimethylaminoethylmethacrylate or Polyquaternium-11) of ISP, New Jersey, U.S.A., or Abil Quat 3272 (diquaternary polydimethylsiloxane or Quaternium-80) of Th. Goldschmidt AG, Essen, Germany, or UcarePolymer JR400 (polyquaternium-10) of Amercol, New Jersey, U.S.A., or Cosmedia Guar C 261 (hydroxypropyl guar hydroxypropyltrimonium chloride) of Henkel, Düsseldorf, Germany, or Jaguar C13S (guar hydorxypropyltrimonium chloride) of Rhône Poulenc, France, or Merquat 550 (Polyquaternium 7) of Chemviron SA, or Lamequat L (lauryidimonium hydroxypropyl hydrolyzed collagen) of Grünau/Henkel. The cationic polymers can be employed in the compositions of the invention in concentrations of from 0.01 to 3 percent by weight.

Besides the above-described ingredients the hair care composition according to the invention can include still other ingredients, which are normally used in hair care compositions. These ingredients include consistency-imparting substances and thickeners, such as agar-agar, guar gum, alginates and xanthen gum or esters of ethoxylated polyols and fatty acids, such as polyglyceryl (2) polyoxyethylene(4)stearate, and oils and waxes, such as triglycerides of plant or animal origins, spermaceti, beeswax, montan wax, paraffin oils, petrolatum (Vaseline®), wool fat, fruit waxes and other plant waxes.

Publications and monographs known to those skilled in the art can be referred to for information regarding the conventional cosmetic additives. For example, the publication by Andreas Dornsch, “Cosmetic Perparations (Die kosmetischen Präparate)”, Chem. Industrie Verlag (Press), H. Ziolkowsky KG, Augsburg, Edition, Volume 2, pp. 212-230, 1992.

The hair cosmetic compositions according to the invention can have a pH between 1.0 and 9.0, preferably between 4.0 to 7.0, and especially preferably between 4.5 and 5.6. The pH can be adjusted, as desired, by a suitable cosmetically compatible acid. Citric acid, tartaric acid, lactic acid, adipic acid, glyoxylic acid, gluconic acid, malic acid or phosphoric acid are suitable for this purpose.

In the method of treating an existing case of dandruff formation or of preventing or reducing the likelihood of dandruff formation according to the invention the desired anti-dandruff action is achieved in an advantageous manner, when the compositions according to the invention, especially in the form of a shampoo, are brought into contact with the skin surface on the head during hair washing. When a hair tonic, care composition or rinse is employed the appropriate composition is applied daily or at least once a week over a time interval of days, weeks or months or continuously. The duration and frequency of usage depend on the type, form and extent of the scalp dandruffs.

The following examples should illustrate the subject matter of the invention in more detail. The stated numerical values are percentages by weight in relation to 100% of the pure substance.

### EXAMPLES

The following exemplary compositions can be prepared according to the known methods. The raw materials are generally known and are obtainable commercially. Composition A is the sole example of an anti-dandruff composition according to the invention. Compositions B to F are comparative compositions according to the prior art.

TABLE I
ANTI-DANDRUFF SHAMPOOS ACCORDING TO THE INVENTION
AND THE PRIOR ART

| Ingredient | A | B | C | D | E | F |
|---|---|---|---|---|---|---|
| Sodium laureth sulfate | 9.80 | 9.80 | 9.80 | 9.80 | 9.80 | 9.80 |
| Cocamidopropyl | 1.50 | 1.50 | 1.50 | 1.50 | 1.50 | 1.50 |
| Betaine | | | | | | |
| Climbazole | 0.50 | 2.0 | 0.5 | 2.0 | — | — |
| Piroctone olamine | 0.50 | 2.0 | 0.5 | — | 2.0 | |
| Zinc pyrithione | 2.00 | — | — | — | — | 2.0 |
| Perfume | 0.40 | 0.40 | 0.40 | 0.40 | 0.40 | 0.40 |
| Sodium chloride | 2.80 | 2.80 | 2.80 | 2.80 | 2.80 | 2.80 |
| Preservative | 0.50 | 0.50 | 0.50 | 0.50 | 0.50 | 0.50 |
| Water | To 100 | To 100 | To 100 | To 100 | To 100 | To 100 |

The composition A according to the invention had significantly better anti-dandruff action than the comparative compositions B to F according to the prior art.

The disclosure in German Patent Application 101 11 288.2 of Mar. 9, 2001 is incorporated here by reference. This German Patent Application describes the invention described hereinabove and claimed in the claims appended hereinbelow and provides the basis for a claim of priority for the instant invention under 35 U.S.C. 119.

While the invention has been illustrated and described as embodied in an anti-dandruff composition and method for preventing the occurrence of dandruff formation or for treating an existing case of dandruff formation on skin covered by hair, especially scalp skin, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. An anti-dandruff composition comprising a combination of climbazole, zinc pyrithione and piroctone olamine.

2. The anti-dandruff composition as defined in claim 1, further comprising at least one synthetic chemical or natural anti-dandruff agent besides said climbazole, said zinc pyrithione and said piroctone olamine.

3. The anti-dandruff composition as defined in claim 1, further comprising at least one synthetic chemical or natural anti-dandruff agent selected from the group consisting of salicylic acid, sulfur, tar preparations, undeconoic acid derivative compounds, hinokitol, burdock root extracts, poplar extracts, burned nettle extracts, walnut shell extracts, birch extracts, willow bark extracts and arnica extracts.

4. The anti-dandruff composition as defined in claim 1, 2 or 3, in the form of a shampoo, a douche bath, a douche gel, a gel, a care composition, a rinse, a hair tonic, a hair spray, a foam, a fixing agent, a deodorant or a shaving water.

5. The anti-dandruff composition as defined in claim 1, 2 or 3, further comprising at least one cosmetic additive ingredient selected from the group consisting of thickeners, anionic surfactants, amphoteric surfactants, zwiterrionic surfactants, nonionic surfactants, alcohols, solvating agents, stabilizers, perfumes, dyestuffs, hair conditioning substances and hair care substances, preservatives, inorganic or organic acids, consistency-imparting agents, light-protective agents and UV-filtering polymers.

6. The anti-dandruff composition as defined in claim 1, 2 or 3, containing from 0.03 to 20 percent by weight of said combination and from 0.01 to 10 percent by weight of each of said climbazole, said zinc pyrithione and said piroctone olamine.

7. The anti-dandruff composition as defined in claim 1, 2 or 3, further comprising water and having a pH of from 4.0 to 7.0.

8. A method of eliminating or alleviating existing dandruff formation on a surface of a body of a person, said surface having hair growing thereon, said method comprising applying an effective amount of a combination of climbazole, zinc pyrithione and piroctone olamine for elimination or alleviation of said dandruff formation to said surface of said body and said hair growing thereon.

9. The method as defined in claim 8, wherein said surface is on a scalp of said body, on a beard region of said body, on a shoulder region of said body or in an intimate region of said body.

10. A method of preventing or reducing occurrences of dandruff on a body surface of a person, said surface having hair growing thereon, said method comprising applying an effective amount of a combination of climbazole, zinc pyrithione and piroctone olamine to said body surface for prevention or reduction of said occurrences of said dandruff.

11. The method as defined in claim 10, wherein said body surface is located on a scalp of said person, on a beard region of said person, on a shoulder region of said person or in an intimate region of said person.

12. A method of making a preparation for treating dandruff, said preparation containing a combination of climbazole, zinc pyrithione and piroctone olamine.

13. The method as defined in claim 12, wherein said preparation contains from 0.03 to 20 percent by weight of said combination and from 0.01 to 10 percent by weight of each of said climbazole, said zinc pyrithione and said piroctone olamine.

14. The method as defined in claim 12, wherein said preparation comprises at least one cosmetic additive ingredient selected from the group consisting of thickeners, anionic surfactants, amphoteric surfactants, zwiterrionic surfactants, nonionic surfactants, alcohols, solvating agents, stabilizers, perfumes, dyestuffs, hair conditioning substances and hair care substances, preservatives, inorganic or organic acids, consistency-imparting agents, light-protective agents and UV-filtering polymers.
